(19) United States
(12) Patent Application Publication
(10) Pub. No.: US 2013/0150621 A1
(43) Pub. Date: Jun. 13, 2013

(54) PROCESSES FOR PURIFICATION OF SUCCINIC ACID VIA DISTILLATION

(71) Applicant: BioAmber S.A.S., Bazancourt (FR)

(72) Inventors: Roger L. Bernier, Montreal (CA); Dilum Dunuwila, Princeton, NJ (US); Michael C.M. Cockrem, Madison, WI (US); Olan S. Fruchey, Hurricane, WI (US); Brian T. Keen, Pinch, WV (US); Brooke A. Albin, Charleston, WV (US); Bernard D. Dombek, Charleston, WV (US); Nye A. Clinton, Hurricane, WV (US)

(73) Assignee: BioAmber S.A.S., Bazancourt (FR)

(21) Appl. No.: 13/712,358

(22) Filed: Dec. 12, 2012

Related U.S. Application Data
(60) Provisional application No. 61/569,920, filed on Dec. 13, 2011.

(51) Int. Cl. C07C 51/44; C07C 51/43

(52) U.S. Cl. CPC C07C 51/44; C07C 51/43
USPC 562/593; 203/91; 203/39

(57) ABSTRACT

Processes for removing color bodies from crude succinic acid comprising distillation of crude succinic acid and collecting the distillate in a water-containing receiver. The color bodies substantially remain in the distillation bottoms and the purified succinic acid in the water-containing receiver is substantially free of color bodies.

### RELATED APPLICATION

This nonprovisional application is based upon and claims the benefit of priority from U.S. Application No. 61/569,920, filed Dec. 13, 2011, the disclosure of which is incorporated herein by reference.

### TECHNICAL FIELD

This disclosure relates to processes for the purification of crude succinic acid (SA), particularly fermentation derived SA, by distillation. It is particularly related to removal of color bodies from crude SA to yield SA having little or no visible color.

### BACKGROUND

SA can be produced via fermentation as the diammonium salt. For example, processes for making SA from ammonium salts are described in US 2011/0237831 and US 2011/0272269. The fermentation process results in impurities, such as color bodies, in the isolated fermentation broth and product.

The diammonium succinate (DAS) salt containing broth can be subjected to reactive distillation to remove the first ammonia yielding a monoammonium succinate (MAS) solution which upon concentration and crystallization will yield (after filtration) solid monoammonium succinate which is essentially free of the other by-products present in the fermentation broth. Dissolution of this solid monoammonium succinate in water and subjection to reactive distillation under pressure will remove the second ammonia. Concentration of the solution followed by crystallization and filtration (or centrifugation) will yield solid succinic acid. The recovered solids can, however, contain some color bodies and exhibit an odor.

Alternatively, the diammonium containing broth can be subjected to reactive distillation under conditions that remove both ammonias yielding succinic acid directly. Concentration of the direct distillation residue and crystallization followed by filtration tends to yield succinic acid solids which are more highly colored than the two step process which removes most of the color bodies via the monoammonium succinate recovery step.

The succinic acid produced by both processes may be dark red or brown in color and need further purification to yield a white odorless solid which would be acceptable for use in the production of polymers.

Accordingly, it would be desirable to have an efficient process for the purification of crude SA that does not use recrystallization or carbon for removal of color bodies.

### SUMMARY

We provide a process for removing color bodies from crude SA comprising providing crude SA; distilling the crude SA such that the color bodies substantially remain in the distillation bottoms; and recovering in a water-containing receiver purified SA substantially free of color bodies.

We also provide a process for removing color bodies from crude SA comprising providing crude SA; sublimating the crude SA in a sublimation vessel such that the color bodies substantially remain as residue in the sublimation vessel; and recovering purified SA substantially free of color bodies.

### BRIEF DESCRIPTION OF THE DRAWINGS

FIG. 1 is a block diagram of one example of a process for purifying SA to remove fermentation-derived color bodies from crude SA with two stage reactive distillation.

FIG. 2 is a block diagram of one example of a process for purifying SA to remove fermentation-derived color bodies from crude SA with one stage reactive distillation.

FIG. 3 is a block diagram of an example of distillation column and water-containing receiver.

### DETAILED DESCRIPTION

It will be appreciated that at least a portion of the following description is intended to refer to representative examples of processes selected for illustration in the drawings and is not intended to define or limit the disclosure, other than in the appended claims.

Conventional methods of removal of color bodies from SA use recrystallization and carbon treatment. Recrystallization in conjunction with carbon treatment can improve the quality of the SA, but this may take several stages to remove the color and the odor. These crystallization steps increase cost and processing time and result in decreased yield of product.

Distillation was believed to be unsuitable because it was expected that the color bodies would be carried into the receiver with the distillate. Moreover, when SA is melted and then subjected to a vacuum distillation under conventional methods, it would be expected to yield a distillate which is a mixture of SA and succinic anhydride. Upon cooling the distillate forms a large solid mass which is difficult to remove from the receiver. While the solid mass could be flaked, it would not be pure SA.

We discovered that vacuum distillation of molten crude SA into a water-containing receiver leaves substantially all of the color bodies behind in the distillation bottoms and yields a white slurry of substantially purified SA in the receiver. When the distillation receiver contains water and acts as the condensing agent, the vapor from the distillation rapidly reacts with the water forming a slurry of solid, white SA in water.

Not to be bound by any one theory, we believe that the distillation occurs through the following process. SA is a solid at room temperature and has a melting point of about 185° C. and a boiling point of about 235° C. at 760 torr and will undergo sublimation under a vacuum of 2 torr at 156° C. Molten SA tends to lose water and be partially converted into succinic anhydride. Succinic anhydride has a melting point of 120° C. and a boiling point of 261° C. at 760 torr. Succinic anhydride sublimes at 2 torr and 90° C. Thus, the molten SA in the distillation flask first loses water and is converted to succinic anhydride. Because succinic anhydride has a lower boiling point than SA, it distills from the distillation flask into the water containing receiver where the anhydride immediately reacts with water reforming SA.

Substantially all of the crude SA distilled may be dehydrated to succinic anhydride in the process. However, it is also possible that the vapor is a mixture of SA and succinic anhydride (along with some water) and not pure succinic anhydride. In such a case, a portion of the crude SA would dehydrate to succinic anhydride while a portion of the crude SA remains SA. In either case, all measurable amounts of the succinic anhydride formed during the process convert to form purified SA in the water-containing receiver.

One aspect of the distillation is the presence of excess water in the receiver to convert the anhydride back to succinic acid. Preferably, the amount (by weight) of water in the receiver is greater than or equal to the amount (by weight) of crude SA in the distillation pot. Preferably, the amount of water is about twice the amount by weight of crude SA being distilled, but may be more or less than that amount. There is no upper limit to the amount of water in the water-containing receiver for the purification process, although the recovery and concentration of the SA is more difficult as the amount of water increases. However, when the amount of water is too low, the purified SA is a thick slurry that is difficult to process. Accordingly, the amount of water (by weight) in the water-containing receiver may be greater than or equal to 1, 1.5, 2, 2.5, 3, 3.5 or 4 times as much as the amount (by weight) of crude SA being distilled.

Preferably, the crude SA is melted and in a molten state before distillation. The distillation process operates with both wet and/or dry crude succinic acid solids as feed. The solid crude SA may contain less than 2%, less than 1.5%, less than 1% water (by weight), but may be in greater amounts of water as well. Thus, a crude wet SA centrifuge cake obtained from crystallization does not need to be dried before distillation.

The crude SA may be obtained by fermentation, but can be also obtained by adding a stoichiometric amount of strong mineral acid (e.g. sulfuric acid or the like) to a succinate salt containing broth or obtained from the electrodialysis of a succinate salt. Furthermore, the crude SA acid can also be obtained from hydrogenation/hydrolysis of petroleum based maleic anhydride.

Preferably, crude SA is obtained from a clarified DAS-containing fermentation broth by optionally adding an ammonia separating and/or water azeotroping solvent to the broth, distilling the broth at a temperature and pressure sufficient to form an overhead that comprises water and ammonia, and a liquid bottoms that comprises SA, and at least about 20 wt % water, cooling and/or evaporating the bottoms to attain a temperature and composition sufficient to cause the bottoms to separate into a liquid portion and a solid portion that is substantially pure SA, and separating the solid portion from the liquid portion. Alternatively, SA may be obtained from a clarified MAS-containing fermentation broth by optionally adding an ammonia separating and/or water azeotroping solvent to the broth, distilling the broth at a temperature and pressure sufficient to form an overhead that comprises water and ammonia, and a liquid bottoms that comprises SA, and at least about 20 wt % water, cooling and/or evaporating the bottoms to attain a temperature and composition sufficient to cause the bottoms to separate into a liquid portion and a solid portion that is substantially pure crude SA, and separating the solid portion.

The crude SA, though containing color bodies, may be free or essentially free of other carboxylic or organic acids, ammonium salts and/or fermentation by products. Preferably, the crude SA has a purity of greater than or equal to 85%, 90%, 95%, 97% or 99% or even more.

The resulting purified SA is preferably essentially free from succinic anhydride, color bodies or other impurities. In other words, succinic anhydride present in the vapor substantially hydrolyzes to SA. Surprisingly, the fermentation-derived color bodies in the crude succinic acid do not distill with the succinic acid and remain in the distillation bottoms. The use of water as the condensing agent also results in formation of small particles of succinic acid which are easily removed from the receiver (i.e. eliminates the large solid mass formation).

Furthermore, the slurry in the receiver can be filtered with recycle of the mother liquor back to the receiver, thus allowing recovery of the solid SA. The wet solids can have a minor odor which is eliminated after vacuum drying. Alternatively, the slurry may be heated, crystallized, and then dried.

Preferably, the purified SA has a Yellowness Index in methanol less than 5, more preferably less than 3, more preferably less than 2, more preferably less than 1.5, more preferably less than 1. Purified SA of such low Yellowness Index can be obtained from crude SA having a Yellowness Index in methanol of more than 10, including more than 11, more than 12, and more than 13. SA substantially free of color bodies means that the SA appears white upon visual inspection. Preferably, SA substantially free of color bodies has a Yellowness Index of less than about 5.

This distillation residue may also contain some succinic acid which can be recycled back to the ammonia removal distillation step, after dissolution in water. Recycle of SA remaining in the distillation bottoms allows removal of the color bodies via the first stage (i.e. DAS to MAS reaction step) mother liquor purge and recovery of the SA content as monoammonium succinate solids. Distillation with full recycles have essentially no loss of SA. Near quantitative recovery of contained succinic acid is possible.

The water-containing receiver may be loaded with clean or purified water. However, the water in the water-containing receiver may be unpurified water or may be water recycled from another process step. The water may or may not contain one or more solutes, solvents or contaminants.

The distillation process can be performed either in batch or continuous mode.

In batch mode, a distillation pot is charged with solid crude SA. The solids are heated, forming a liquid melt in the distillation pot. After melting, a vacuum is applied to the distillation system and vapor is then carried to the water containing receiver. When the distillation is complete, the vacuum is broken and residue in the distillation pot is then dissolved in hot water and discharged for recycle to the DAS to MAS reactor.

In continuous mode, the system may have two feed vessels. Solid succinic acid is charged to one of the feed vessels and then melted. The molten succinic acid is then fed continuously to the distillation system. While the first feed vessel is feeding the system, the second feed vessel is charged and succinic acid melted. When the first feed vessel is empty the second feed vessel is brought on line to feed the distillation apparatus and then the first feed vessel is again charged with solid succinic acid and melted. This allows the distillation tower to operate in a continuous mode with molten feed.

The distillation tower can be either a one stage flash or a multistage column. The preferred mode of operation is a one stage flash. This one stage flash can be either a wiped film evaporator, thin film evaporator, falling film evaporator, thermosiphon reboiler flasher, forced circulation reboiler flasher or the like.

The transfer line between the reboiler and the water containing receiver should be kept hot to prevent fouling (i.e. solids formation via solidification). This line should be traced and insulated so that the walls remain at a temperature above the melting point of succinic acid. In continuous mode, the vapor can contain some water as well as succinic acid and succinic anhydride (e.g. the water comes from the dehydration of the acid forming the anhydride). Therefore, for ease of operation the wall temperature should be held above about 200° C.

The distillation can be operated at any pressure, however, the preferred pressure range is about 500 torr to about 1 torr and a most preferred pressure range is about 100 torr to about 50 torr.

The water containing receiver can be operated without circulation or agitation. However, it is preferred that the receiver have a circulation device. Circulation via stirring or pumping is preferred. Furthermore, the receiver can be operated as a quench condenser/receiver (i.e. the slurry is recirculated and sprayed to the top of the receiver like a shower).

The temperature of the water in the receiver is not critical, however, temperatures below about 30° C. are preferred. The temperature of the water can be maintained by using either a heat exchanger and pump around loop or a cooling jacket on the receiver.

The slurry in the receiver can be either filtered or centrifuged to remove the solids. Alternatively, the slurry in the receiver can be pumped into a separate vessel and heated to dissolve the solids. This hot solution can then be cooled to crystallize out the SA. After separation and drying pure SA crystals are obtained.

Addition of a dilute sodium hydroxide solution to the top of the reactive distillation towers is optional. This sodium hydroxide addition should be less than about 5% of the total ammonium ion present, preferably less than about 2% and most preferably less than about 1%. This sodium hydroxide addition tends to assist in ammonia removal by raising the pH of the solution.

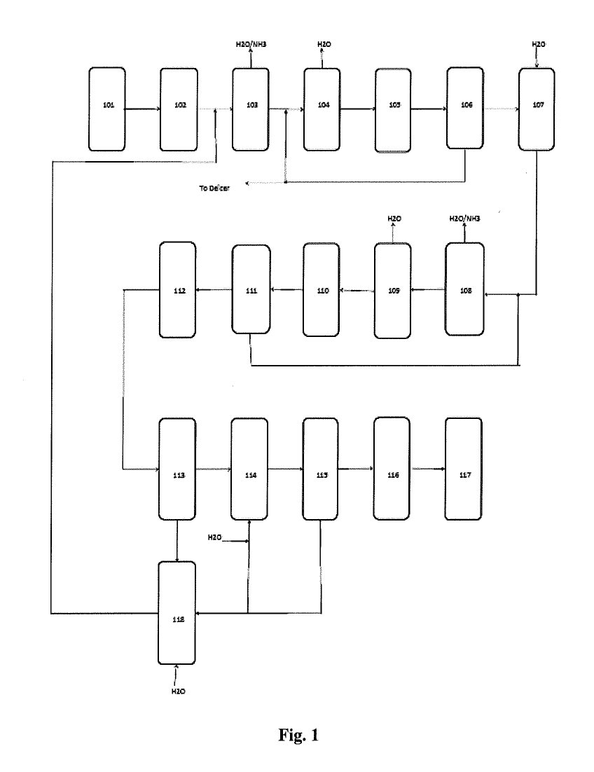

Our processes may be appreciated by reference to FIG. 1, which shows in block diagram form one representative example of our methods. Block 101 is a storage vessel which holds a diammonium succinate containing fermentation broth. The broth is sent to block 102 where it is filtered, yielding clarified broth. This clarified broth is then fed to a reactive distillation tower (block 103) along with optionally additional water. Some of the contained ammonia and water are removed overhead yielding a monoammonium succinate residue stream which is concentrated via flashing (block 104). The residue from block 104 is crystallized in block 105 and the solid monoammonium succinate recovered by centrifugation in block 106 with recycle of a portion of the mother liquor back to the concentrator in block 104 and the rest of the mother liquor being purged to remove by-products (optionally used to make deicer solutions). The solid monoammonium succinate is dissolved in water in block 107 forming a solution which is then fed to block 108 where more water and ammonia are removed via reactive distillation. The distillation residue is then concentrated in block 109 via flashing and the concentrated solution is allowed to crystallize in block 110. The solid succinic acid is separated via centrifugation in block 111 and the mother liquor is recycled back to the reactive distillation step in block 108. The wet solids from the centrifuge are then sent to succinic acid feed vessel (block 112) where they are heated and melted. The molten crude succinic acid is then fed to a thin film evaporator (block 113) where it is distilled under vacuum into a stirred cold water containing receiver (block 114). The slurry in the receiver is then fed to a centrifuge (block 115) where the solid succinic acid is separated and then dried in a vacuum drier (block 116) and placed in product storage (block 117). The residue from the thin film evaporator (block 113) is then dissolved in a portion of the centrifuge (block 115) mother liquor in block 118 and the solution recycled back to the first reactive distillation step (block 103). The rest of the mother liquor is directly recycled back to the receiver (block 114).

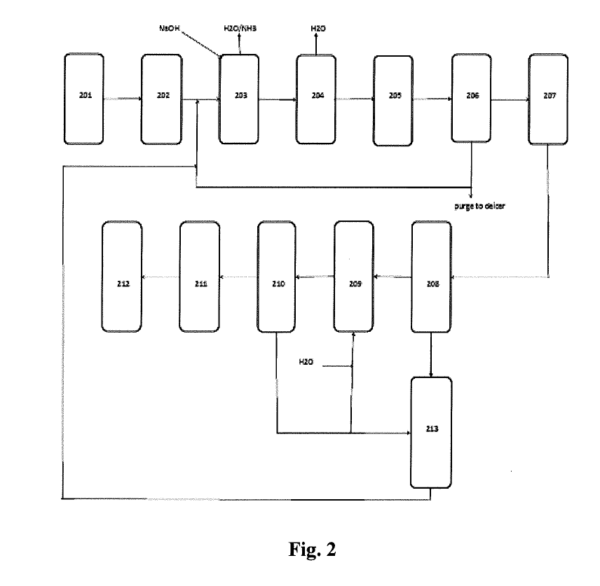

The process in FIG. 1 represents a two stage reactive distillation process for conversion of diammonium succinate into succinic acid. Another example of our process is a one stage reactive distillation process like the one shown in FIG. 2. Block 201 is a storage vessel which holds a diammonium succinate containing fermentation broth. The broth is sent to block 202 where it is filtered yielding clarified broth. This clarified broth is then fed to a reactive distillation tower (block 203) along with optionally additional water and/or dilute sodium hydroxide solution. Some of the contained ammonia and water are removed overhead yielding a succinic acid containing residue stream which is concentrated via flashing (block 204). The residue from block 204 is crystallized in block 205 and the solid succinic acid recovered by centrifugation in block 206 with recycle of a portion of the mother liquor back to the reactive distillation step block 203 and the rest of the mother liquor being purged to remove by-products (optionally used to make deicer solutions). The wet solids from the centrifuge are then sent to succinic acid feed vessel (block 207) where they are heated and melted. The molten crude succinic acid is then fed to a thin film evaporator (block 208) where it is distilled under vacuum into a stirred cold water containing receiver (block 209). The slurry in the receiver is then fed to a centrifuge (block 210) where the solid succinic acid is separated and then dried in a vacuum drier (block 211) and placed in product storage block 212. The residue from the thin film evaporator (block 208) is then dissolved in a portion of the centrifuge (block 210) mother liquor in block 213 and the solution recycled back to the first reactive distillation step (block 203). The rest of the mother liquor is directly recycled back to the receiver (block 209).

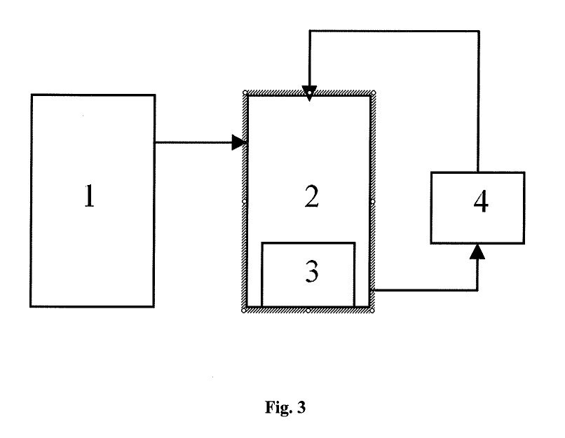

FIG. 3 shows a distillation column 1 and water-containing receiver 2 with optional stirrer 3 and pump 4, representing a method for distilling molten succinic acid and recovering the distilled succinic acid in water.

The fermentation broths used in the above described processes, also shown in FIGS. 1 and 2, can contain either diammonium succinate and/or monoammonium succinate along with other by-product ammonium salts such as acetate, formate, lactate and the like. Both processes lead to the isolation of high quality SA from a succinate containing fermentation broth.

A process for removing color bodies from crude SA comprise, consist, or consist essentially of the steps of the distillation processes described herein. For example, the steps of recrystallization and carbon treatment or other means of removing color bodies are not necessary.

We have described only two of the many possible examples of the process which could be used by one skilled in the art. However, each example involves a distillation of a broth derived crude succinic acid (either wet or dry) into a receiver containing water yielding high quality white succinic acid after recovery and drying.

### EXAMPLES

The processes are illustrated by the following non-limiting, representative examples.

### Example 1

A bulb to bulb distillation apparatus was assembled using a 500 mL single neck round bottom flask as the feed pot and a 250 mL round bottom flask with a 90° side arm at the mid-point of the flask as the receiver. The two flask were connected by a glass tube (−6 inches long and had two 90° bends) which was wrapped with electrical heating tape. The receiver side arm was fitted with a short tube in a tube condenser which was topped with a vacuum adaptor which was connected to a vacuum pump system. The receiver was placed in an ice bath and stirred with a magnetic stirrer. The feed pot was heated with a clamshell heating mantle and stirred with a magnetic stirrer. This apparatus was used for all of the succinic acid acid distillations.

The receiver was filled above the side arm with water and 50 g of crude brown succinic acid (Yellowness index 13.1 in methanol) was placed in the feed pot. The electrical heating tape was brought to 250° C. (outside wall temperature). The contents of the feed pot were then heated to melt the crude succinic acid. Once all of the solids had melted a vacuum of 100 torr was applied and material began distilling over to the receiver where white solids appeared in the stirred liquid. After most of the liquid in the feed pot had been distilled, the vacuum was broken and the power to the heating mantles and electrical tape was turned off. The white slurry in the receiver was filtered yielding 25.5 g of solids. After drying under vacuum, HPLC analysis indicated that only 0.002% succinimide impurity was present in the succinic acid. Yellowness index for the product was 0.76 (i.e. very white material).

### Example 2

The bulb to bulb distillation apparatus described in Example 1 was used for this experiment.

The receiver was filled above the side arm with water and 50 g of crude brown succinic acid (Yellowness index 13.1 in methanol) was placed in the feed pot. The electrical heating tape was brought to 250° C. (outside wall temperature). The contents of the feed pot were then heated to melt the crude succinic acid. Once all of the solids had melted a vacuum of 50 torr was applied and material began distilling over to the receiver where white solids appeared in the stirred liquid. After most of the liquid in the feed pot had been distilled, the vacuum was broken and the power to the heating mantles and electrical tape was turned off. The white slurry in the receiver was filtered yielding 26.1 g of solids. After drying under vacuum, HPLC analysis indicated that only 0.01% succinimide impurity was present in the succinic acid. Yellowness index for the product was 0.71 (i.e. very white material).

### Example 3

100 g of the same crude succinic acid used in Experiment 1 was batch distilled (at 50 torr) using the procedure in Experiment 1. The distilled solids were recovered by filtration and then dried in a vacuum oven. The succinic acid residue left in the distillation flask was dissolved in water and analyzed by HPLC along with the dried solids and mother liquor. Based on the analytical and weights of these three streams we were able to determine that 68.8 g of white succinic acid ended up in the dried solids, 15 g of succinic acid ended up in the mother liquor and 16.1 g of dark brown succinic acid were left in the pot. This gives a 99.9% succinic acid accountability for the run. The total mass accountability for this run was 98.9%.

### Example 4

A pressure distillation column was made using an 8 ft long 1.5″ 316 SS Schedule 40 pipe that was packed with 316 SS Propak packing. The base of the column was equipped with an immersion heater to serve as the reboiler. Nitrogen was injected into the reboiler via a needle valve to pressure. The overhead of the column had a total take-off line which went to a 316 SS shell and tube condenser with a receiver. The receiver was equipped with a pressure gauge and a back pressure regulator. Material was removed from the overhead receiver via blowcasing through a needle valve. Preheated feed was injected into the column approximately ⅔ of the way up the packing via a pump. A dilute sodium hydroxide solution was injected on the top of the packing. Preheated water was also injected into the reboiler via a pump. This column was operated under pressure to give column temperatures greater than 100° C.

The feed to the column was a fermentation derived diammonium succinate broth. It contained 3.9% succinic acid and 0.71% acetic acid, both present as their mixed ammonium/sodium salts. The molar ammonium to sodium ratio was estimated to be ˜10/1. Undetermined small amounts of other carboxylate salts such as formic, pyruvic and fumarate with miscellaneous fermentation residues were also present.

Initially this broth was fed to the column at a rate of 8 mL/min and 0.2% sodium hydroxide solution was fed at 0.2 mL/min. Water was fed to the reboiler at a rate of 5 mL/min. The overhead distillate rate was 9 mL/min and the residue rate was 4.1 mL/min. The column was operated at 120 psig which gave a temperature of 178° C. and the residence time in the column was ˜45 minutes. After ˜12 hrs of operation over a two day period (˜6 hrs per day) the collected residue was then fed back to the column at a rate of 4.1 mL/min along with 0.2 mL/minute of 0.2% sodium hydroxide solution and the reboiler was fed 9.2 mL/min of water with a tails rate of 4.2 mL/min and a distillate rate of 9.2 mL/min. The column was operated at a pressure of 162 psig which gave a temperature of ˜193° C. The column residence time was 45 minutes. After ˜12 hrs of operation over a two day period (˜6 hrs per day) under these conditions the collected residue was then fed back to the column at a rate of 4.3 mL/min along with 0.2 mL/min of 0.2% sodium hydroxide solution and the reboiler was fed water at a rate of 10.5 mL/min. During this operation the distillate rate was 10.5 mL/min and the residue rate was 4.5 mL/min with a residence time of 40 minutes. The column was operated under these conditions for ˜12 hrs over a two day period.

The recovered residue (3199 g) was concentrated under vacuum with a maximum temperature of 88° C. yielding 533 g which was then cooled to room temperature. About 70 g of tan crystals were filtered and air dried by sucking air through the filter funnel. The crystals were tan in color and had a strong odor. The succinic acid distillation apparatus described above was then used to distill these crystals.

The receiver was filled above the side arm with water (˜100 g) and 50 g of the air dried solids were placed in the feed pot. The electrical heating tape was brought to 250° C. (outside wall temperature). The contents of the feed pot were then heated to melt the crude succinic acid. Once all of the solids had melted a vacuum of 50 torr was applied and material began distilling over to the receiver where white solids appeared in the stirred liquid. After part of the liquid in the feed pot had been distilled the vacuum was broken and the power to the heating mantles and electrical tape was turned off. The white slurry in the receiver was filtered yielding 17 g of solids. After drying under vacuum at 75° C., HPLC analysis indicated that 2.9% succinimide impurity was present in the white succinic acid solids which had no odor.

### Example 5

A sample of SA was analyzed for organic impurities by HPLC using refractive index detection and a simple area purity used. Concentrations of known and unknown impurities present were not estimated in this case using response factors typical for known fermentation impurities. It was found that by area only basis, this SA was 99.655% pure.

A sample of around 2 gram of succinic acid was heated at 180° C. in a tube with one end located in a heating block for at least 2 hours and the other end exposed to the atmosphere and thus cooler. The condensed biobased succinic acid was collected and analyzed as above. The purity had increased to 99.9204% by HPLC. Visual inspection shows increased purity in terms of color. This corresponds to a measured area level of impurities of 795 ppm.

Any US patents, published US patent applications or publications referred to herein are incorporated in the entirety.

Although our processes have been described in connection with specific steps and forms thereof, it will be appreciated that a wide variety of equivalents may be substituted for the specified elements and steps described herein without departing from the spirit and scope of this disclosure as described in the appended claims.

1. A process for removing color bodies from crude SA comprising:
a) providing crude SA;
b) distilling the crude SA such that the color bodies substantially remain as distillation bottoms resulting from a distillation vessel; and
c) recovering in a water-containing receiver purified SA substantially free of color bodies.

2. The process of claim 1, wherein the crude SA is molten.

3. The process of claim 1, wherein an amount of water in the water-containing receiver is greater than or equal to the amount of crude SA.

4. The process of claim 3, wherein an amount of water in the water-containing receiver is greater than or equal to twice the amount of the crude SA.

5. The process of claim 1, wherein temperature of the water in water-containing receiver is below about 30° C.

6. The process of claim 1, wherein the water-containing receiver is agitated.

7. The process of claim 6, wherein the water-containing receiver is agitated with a stirrer.

8. The process of claim 6, wherein the water-containing receiver is agitated by a pump around loop.

9. The process of claim 8, wherein a return line of the pump around loop is sprayed into the water-containing receiver forming a quench zone.

10. The process of claim 1, wherein a portion SA dehydrates to succinic anhydride.

11. The process of claim 1, wherein step (b) is conducted under vacuum.

12. The process of claim 11, wherein absolute pressure is about 500 to about 1 torr.

13. The process of claim 11, wherein absolute pressure is about 100 to about 50 torr.

14. The process of claim 1, wherein a Yellowness index in methanol of the crude SA is greater than about 10.

15. The process of claim 14, wherein a Yellowness index in methanol of the crude SA is greater than about 12.

16. The process of claim 1, wherein a Yellowness index in methanol of purified SA is less than about 5.

17. The process of claim 16, wherein the Yellowness index in methanol of the purified SA is less than about 1.

18. The process of claim 1, wherein step (b) is performed in batch or continuous mode.

19. The process of claim 1, further comprising:
d) separating the purified SA.

20. The process of claim 1, wherein purified SA is a white slurry.

21. The process of claim 1, wherein the crude SA is fermentation derived.

22. A process for removing color bodies from crude SA comprising:
a) providing crude SA;
b) sublimating the crude SA in a sublimation vessel such that the color bodies substantially remain as residue in a sublimation vessel;
c) recovering purified SA substantially free of color bodies.
